(19) United States
(12) Patent Application Publication
(10) Pub. No.: US 2025/0250530 A1
(43) Pub. Date: Aug. 7, 2025

(54) PROCESS FOR FERMENTING YEAST

(71) Applicants: DSM IP ASSETS B.V., Heerlen (NL); Technische Universiteit Delft, Delft (NL)

(72) Inventors: Wouter Adrianus VAN WINDEN, Den Haag (NL); Hendrik Jan NOORMAN, Delft (NL); Robert MANS, Delft (NL); Rob Andreas Jacobus VERLINDEN, Den Haag (NL); Stefaan BREESTRAAT, Rotterdam (NL)

(21) Appl. No.: 19/087,650

(22) Filed: Mar. 24, 2025

Related U.S. Application Data
(63) Continuation of application No. 18/546,047, filed on Aug. 10, 2023.

(30) Foreign Application Priority Data
Feb. 15, 2021 (EP) 21157235.9
Oct. 1, 2021 (EP) 21200519.3

(51) Int. Cl. C12M 1/36; C12M 1/00; C12N 1/16; C12P 21/00; C12R 1/645

(52) U.S. Cl. CPC C12M 41/48; C12M 43/06; C12M 47/18; C12N 1/165; C12P 21/00; C12R 2001/645

(57) ABSTRACT

The present invention relates to a process for fermenting Yarrowia in a bioreactor, comprising cultivating Yarrowia in a medium, and adding a feed comprising formic acid and a sugar to the medium in a molar ratio of formic acid to sugar of 1 to 13 mol formic acid/mol monosaccharide.

### CROSS-REFERENCE TO RELATED APPLICATIONS

This application is a continuation application of U.S. application Ser. No. 18/546,047, filed 10 Aug. 2023, which is a National Stage entry of International Application No. PCT/EP2022/053502, filed 14 Feb. 2022, which claims priority to European Patent Application No. 21200519.3, filed 1 Oct. 2021, and European Patent Application No. 21157235.9, filed 15 Feb. 2021, the entire content of each of which is incorporated herein by reference.

### BACKGROUND

### Field

The present invention relates a process for fermenting a yeast in the presence of formic acid and glucose.

### Description of Related Art

Yarrowia lipolytica is a yeast species which is widely used in industrial fermentation processes. Fermentation processes are usually energy intensive and CO₂ (carbon dioxide) is emitted as a result of energy use as well as metabolism of the microorganisms (so-called biogenic CO₂). There is a need to reduce CO₂ emissions because of its effect on climate change. WO2020193516 discloses a process for cultivating a microorganism capable of utilizing an organic feedstock, wherein CO₂ is captured and reduced to an organic feedstock such as formic acid.

It is known that when the yeasts Candida utilis and Hansenula polymorpha are cultivated in the presence of formate and glucose an enhanced biomass cell yield on glucose was obtained as compared to cultivation on glucose alone. However, Saccharomyces cerevisiae did not show an enhanced biomass cell yield when grown in the presence of formate and glucose (see Babel, W., et al. (1983). Archives of Microbiology, 136 (3), 203-208, and Bruinenberg, P. M., et al., (1985), Archives of Microbiology, 142 (3), 302-306). In the cited studies, formate (i.e. the conjugate base of formic acid) was fed to the cultures of the yeast species. The glucose to formate ratios at which increased biomass yields were obtained for Candida and Hansenula did not correspond to theoretical predictions. More formate was needed to obtain increased yields than predicted, which was attributed to energetic costs for formate transport into the cell, the uncoupling effect of formate, and alternative formate oxidizing systems that did not generate metabolic energy.

The present invention relates to an improved process for fermenting a yeast of Yarrowia sp.

### BRIEF DESCRIPTION OF THE DRAWINGS

FIG. 1. In FIG. 1, the substrate sugar is introduced via conduits 13 into bioreactor 1. The bioreactor 1 provides the conditions allowing fermentation of Yarrowia yeast cells capable of utilizing formic acid as co-substrate. The produced product, like the Yarrowia yeast cells or a compound of interests produced by the Yarrowia yeast, leaves the bioreactors 1 via conduits 14. CO₂ formed in the bioreactor 1 is introduced into a CO₂ capture unit 2 via conduits 15. In reduction unit 3 the CO₂ is reduced to formic acid, utilizing H₂. The formic acid is introduced into bioreactor 1 via conduits 10, enabling fermentation of the Yarrowia yeast that is capable to utilize the organic feedstock. The advantage is that biogenic CO₂ is not released into the environment, and the amount of sugar needed for the fermentation process is reduced. The H₂ needed for reduction of CO₂ into formic acid can be introduced into reduction unit 3 and can be sourced from a supplier. An electrolysis unit 4 can electrolyze water into H₂ and O₂. The H₂ can be introduced into reduction unit 3 via conduit 11. The O₂ originating from the electrolysis of water can be introduced into bioreactor 1 via conduit 12. Alternatively, or simultaneously, air can be introduced into bioreactor 1 for aerobic processes.

FIG. 2. Overview of determined biomass yields of Yarrowia lipolytica (gram cell dry weight formed per gram glucose consumed) over the different formic acid to glucose feed ratios. Biomass concentrations used for these calculations were determined from samples taken directly from the bioreactor and ingoing/residual formic acid concentrations were determined via HPLC analysis.

### SUMMARY OF THE INVENTION

The present invention relates to a process for fermenting Yarrowia in a bioreactor, comprising cultivating Yarrowia in a medium, and adding a feed comprising formic acid and a sugar to the medium in a molar ratio of formic acid to sugar of 1 to 13 mol formic acid/mol monosaccharide.

Surprisingly, it was found that an increased biomass yield on sugar was obtained when Yarrowia was fed formic acid in addition to a sugar at a molar ratio of 1 to 13 mol formic acid/mol of monosaccharide.

### DETAILED DESCRIPTION OF A PREFERRED EMBODIMENT

The present invention relates to a process for fermenting Yarrowia in a bioreactor, comprising cultivating Yarrowia in a medium, and adding a feed comprising formic acid and a sugar to the medium in a molar ratio of formic acid to sugar of 1 to 13 mol formic acid/mol monosaccharide.

Surprisingly, it was found that an increased biomass yield on sugar was obtained when Yarrowia was fed formic acid in addition to a sugar at a molar ratio of 1 to 13 mol formic acid/mol monosaccharide, preferably at a molar ratio of 1.5 to 12 mol formic acid/mol monosaccharide, preferably at a molar ratio of 2 to 10 mol formic acid/mol of monosaccharide, preferably at a molar ratio of 2.5 to 9 mol formic acid/mol monosaccharide, preferably at a molar ratio of 3 to 8 mol formic acid/mol monosaccharide, preferably at a molar ratio of 3.5 to 7 mol formic acid/mol monosaccharide, preferably at a molar ratio of 4 to 6 mol formic acid/mol of monosaccharide, preferably at a molar ratio of 4.2 to 5.8 mol formic acid/mol of monosaccharide, preferably at a molar ratio of 4.5 to 5.5 mol formic acid/mol of monosaccharide.

Any suitable sugar may be fed to the medium in a process as disclosed herein, for instance glucose, fructose, sucrose, maltose, maltotriose and/or other oligosaccharide. Preferably, the sugar comprises or is glucose. The molar ratio of formic acid to sugar is calculated on the basis of moles of monosaccharide in the sugar.

A feed comprising formic acid and a sugar may comprise one feed comprising formic acid and a sugar, or one feed comprising formic acid and a second feed comprising a sugar. A person skilled in the art knows how to add a feed comprising formic acid and a sugar to a medium in a process for fermenting Yarrowia as disclosed herein.

A feed comprising formic acid has a pH of 1 to 6, preferably a pH of 1.5 to 5.5, preferably a pH of 2 to 5, or a pH of 2.5 to 4.5, or a pH of 1 to 3. Preferably formic acid in the feed is in the acid form. Preferably, the feed comprising formic acid has a pH which has not been adjusted to the pH of the cultivation medium in a process as disclosed herein. Preferably, the pH of the feed has not been adjusted by using an alkaline titrant, such as potassium or sodium hydroxide. Advantageously, it was found no titrant was needed to adjust the pH of a feed comprising formic acid and a sugar in a process as disclosed herein, resulting in lower salt levels in the fermentation medium as compared to a fermentation process wherein a titrant is used in the feed.

Surprisingly, it was found that no extra titrant, such as an alkaline titrant, was needed to adjust the pH of the cultivation medium when formic acid was fed in process as disclosed at a ratio of formic acid/mol monosaccharide as disclosed herein above.

The process for fermenting Yarrowia may be performed under any suitable conditions, preferably under carbon-limited conditions. A carbon limited condition is defined herein as a condition, wherein the biomass specific growth rate of the yeast is determined by the rate of feeding carbon substrate(s), and no or only low residual levels of carbon substrate(s) are present in the culture at any time during cultivation. The advantage of fermenting Yarrowia under carbon (C)-limited conditions is that a carbon source, such as formic acid and sugar, that is fed to the fermentation is consumed instantaneously by the cell and does not accumulate in the broth. In a process for fermenting Yarrowia as disclosed herein cultivating under carbon-limited conditions prevents that formic acid becomes toxic to the cell.

A process for fermenting Yarrowia is performed in any suitable medium for fermenting Yarrowia, known to a person skilled in the art. The medium in a process as disclosed herein has a pH of from 3 to 7, for instance from 4 to 6.5, for instance a pH from 4 to 6.

The process for fermenting Yarrowia may be performed as any suitable culture, such as a continuous culture or a fed batch culture. A continuous culture may be a chemostat culture. Continuous or chemostat cultures are known to a person skilled in art, and comprise a bioreactor, for instance a chemostat, to which fresh medium is continuously added and culture liquid comprising left over nutrients, microbial cells, such as Yarrowia cells and possibly other products of interest are removed continuously at the same rate to keep the culture volume constant.

The ratio between the rate at which a feed is added to the bioreactor and the volume of the culture, also called the dilution rate, controls the specific growth rate (u) of a microorganism, such as yeast. When the process as disclosed herein is performed as a continuous culture, the continuous culture preferably has a dilution rate of 0.03 h⁻¹ to 0.3 h⁻¹ during steady state, such as a dilution rate of 0.05 h⁻¹ to 0.28 h⁻¹, such as a dilution rate of 0.08 h⁻¹ to 0.25 h⁻¹, such as 0.09 h⁻¹ to 0.21 h⁻¹ during steady state. Steady state is a condition in a continuous culture known to a person skilled in the art, and indicates a condition wherein growth of microbial cells, such as the Yarrowia cells, occurs at a constant specific growth rate, substrate uptake rate, and formation of (by) products and all culture variables, such as volume, dissolved oxygen, pH etc., also remain constant. Preferably, a continuous culture is performed under a carbon limited condition.

In another embodiment a process as disclosed herein is performed as a fed batch culture, which is known to a person skilled in the art, and generally can be defined as a process wherein one or more nutrients or substrates are fed to a bioreactor during fermentation or cultivation and in which products remain in the bioreactor until the end of a culture.

When a process as disclosed herein is performed as a fed batch culture, the fed batch culture comprises a controlled feed rate profile. A controlled feed rate profile and the amount of biomass that is present in the bioreactor at any time during the fed batch culture result in a biomass specific growth rate that varies between the maximum growth rate of the yeast and near-zero growth rates. Preferably, the feed-rate profile during fed-batch results in a carbon-limited condition.

The feed rate profile in a fed batch culture disclosed herein may comprise any suitable feed rate profile known to a person skilled in the art, for instance a linearly increasing, a step-wise increasing feed rate profile, or a feed rate profile comprising a linearly increasing feed or exponentially increasing feed phase, followed by a constant feed phase. Preferably, the feed rate profile comprises an exponential feed phase and a constant feed phase, resulting in a constant biomass specific growth rate during the exponentially increasing feed phase and a decreasing biomass specific growth rate during the constant feed phase. During the exponentially increasing feed phase Yarrowia has a biomass specific growth rate which may range from 0.03 h⁻¹ to 0.3 h⁻¹, preferably from 0.05 h⁻¹ to 0.28 h⁻¹, preferably from 0.08 h⁻¹ to 0.25 h⁻¹, preferably from 0.09 h⁻¹ to 0.21 h⁻¹.

A process for fermenting Yarrowia as disclosed herein is preferably performed at an industrial scale. Preferably, the bioreactor has a volume of at least 10 litres, preferably at least 100 litres, preferably at least 1000 litres, preferably at least 10.000 litres.

In one embodiment a process for fermenting Yarrowia as disclosed herein comprises producing Yarrowia and/or a polypeptide and/or a compound of interest. A compound of interest produced in a process as disclosed herein may for instance be proteins, lipids, steviol glycosides, carotenoids, such as β-carotene, retinoids or other vitamins.

In another embodiment, the Yarrowia yeast comprises at least one polynucleotide coding for a polypeptide of interest or at least one polynucleotide coding for a polypeptide involved in the production of a compound of interest by the yeast. The at least one polynucleotide may be homologous or heterologous to the cells. A person skilled in the art knows how to modify a Yarrowia yeast cell such that it is capable of producing a polypeptide or a compound of interest.

The term “heterologous” as used herein refers to a nucleic acid or polynucleotide or amino acid sequence, or polypeptides not naturally occurring in the yeast cell. In other words, the nucleic acid or polynucleotide, amino acid sequence or polypeptide is not identical to that naturally found in the Yarrowia cell.

A polynucleotide is defined herein as a nucleotide polymer comprising at least 5 nucleotide or nucleic acid units. A nucleotide or nucleic acid refers to RNA and DNA. The terms “nucleic acid” and “polynucleotide sequence” are used interchangeably herein.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes.

The polypeptide may be an enzyme, for instance any suitable hydrolase, such as an esterase, a lipase, for instance a phospholipase, a protease, a cellulase, hemicellulase, or an amylase, or an oxidase, such as a peroxidase, a glucose oxidase, or a monooxygenase, or an isomerase

The compound of interest that is produced in a process as disclosed herein, may for instance be proteins, lipids, steviol glycosides, carotenoids, such as β-carotene, retinoids or vitamins.

A process as disclosed herein comprises fermenting any suitable species of Yarrowia, preferably the Yarrowia sp., is a Yarrowia lipolytica.

In one embodiment a process as disclosed herein, further comprises a step of
i) capturing CO₂ from the bioreactor (1); and
ii) reducing the CO₂ to formic acid in a reduction unit (3); and
iii) feeding at least a part of the formic acid from the reduction unit (3) into the bioreactor (1).

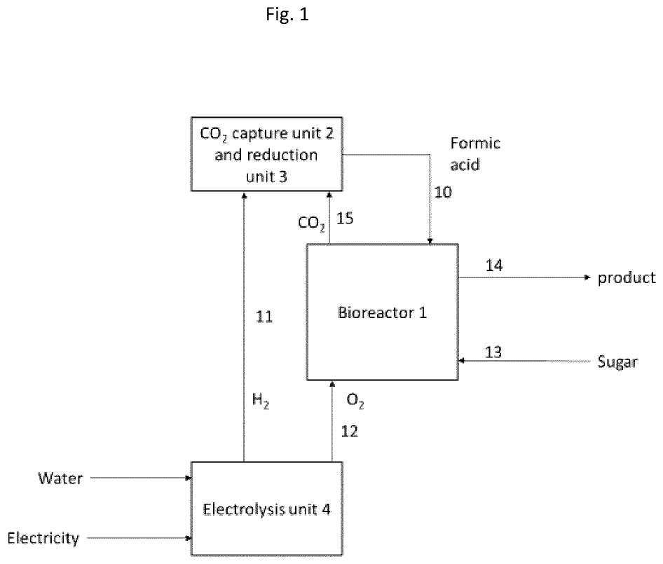

A process wherein CO₂ is captured from a bioreactor is schematically shown in FIG. 1.

Capturing CO₂ from the bioreactor (1) means capturing CO₂ from the off-gas of the bioreactor (1). The captured CO₂ from the off-gas is reduced to formic acid in a reduction unit (3). Capturing and reducing the CO₂ can be carried out by known methods and equipment. Reducing CO₂ may for instance be performed by electrochemical reduction, photoelectrochemical reduction, enzymatic reduction or microbial reduction of the CO₂.

Step (iii) of feeding at least a part of the formic acid from the reduction unit into the bioreactor (1) comprising feeding at least 10% (w/w) of the formic acid from the reduction unit (3). More preferably, feeding at least 20% (w/w), at least 30% (w/w), at least 40% (w/w), at least 50% (w/w), at least 60% (w/w), at least 70% (w/w), at least 80% (w/w), at least 90% (w/w), at least 95% (w/w) or at least 99% (w/w) of formic acid from the reduction unit (3) to the bioreactor (1). More preferably 100% of the formed formic acid is fed into the bioreactor (1). Preferably, the amount of formic acid that is reduced from CO₂ matches the amount of formic acid than can be used for cofeeding into the bioreactor. This provides an improved method that reduces CO₂ emission and can be implemented at lower costs because the needed equipment for CO₂ reduction can be smaller. On the other hand, in view of carbon pricing, it might be advantageous to reduce all CO₂ formed to formic acid. Any surplus of formic acid that cannot be cofed to the bioreactor (1), can be used for other commercial purposes. Hence, the present method may comprise a step of collecting formic acid formed and packaging and/or transporting it.

In another embodiment, the process as disclosed further comprises a step of
iv) electrolyzing water into H₂ and O₂ in an electrolysis unit (4), and feeding at least a part of the H₂ into the reduction unit (3) for reducing the CO₂ to the organic feedstock.

The advantage of electrolyzing water into H₂ and O₂ is that no H₂ needs to be sourced from external sources. The electricity needed for electrolysis unit (4) can advantageously be obtained from surplus electricity of other equipment on site, to provide a sustainable solution for the needed electricity. Alternatively, the electricity is generated from renewable energy, such as solar, wind or hydro energy.

In another embodiment the process as disclosed herein further comprises the step of
(v) feeding at least a part of the O₂ into the bioreactors (1).

The O₂ is the by-product of the electrolysis of water. It can advantageously be used in the bioreactor (1). Furthermore, it was found that all aeration of the bioreactor can be realized by using the O₂ from the electrolysis unit (4). Hence, no O₂ from a different source is needed anymore, which provides a further sustainability improvement. For example, no compressor for air is needed anymore, or a smaller compressor can be used, because the electrolysis unit (4) can deliver the O₂ under pressure that is comparable with the pressure by a conventional compressor. Preferably, the step (iv) of electrolyzing water into H₂ and O₂ in an electrolysis unit (4) is carried out at a pressure within the range of 0.5 to 10 bar, preferably 1 to 8 bar, more preferably 1.5 to 6 bar, most preferably 2 to 4 bar, preferably bar absolute pressure.

In one embodiment, a process as disclosed herein further comprises controlling the fermentation process comprising the steps of:
(vi) selecting at least one process variable of the fermentation process, for which current measured values are determined during the course of the fermentation process;
(vii) comparing a respective current measured value of the at least one selected process variable with a corresponding estimated value of a model variable estimated by a process model for this at least one process variable;
(viii) comparing a variance between the respective current measured value and the corresponding estimated value for the at least one selected process variable with a predetermined threshold value; and
(ix) changing at least one defined model variable employed in the process model when the predetermined threshold value is exceeded by the variance:
   wherein step (vi) to (ix) with a respective changed model variable are executed until the variance is placed below a predetermined threshold value; and optionally
   wherein in cases that, after a predetermined number of repetitions of steps (iv) to (ix) the threshold value is met by the variance, the method is discontinued and a warning is generated as output.

In the bioreactor, CO₂ capturing unit (2), CO₂ reduction unit (3), and/or in the electrolysis unit (4), various environmental and/or process variables can be regulated and controlled for the respective fermentation process, such as pH value, temperature, air supply, CO₂ supply, H₂ supply, oxygen supply, nitrogen supply, formic acid and sugar content and/or mixer settings. However, fermentation processes are biologically complex and very sensitive. Constant close monitoring of the fermentation process is therefore necessary to maintain the corresponding environmental conditions in the bioreactor for a consistent and optimal course of the process and so that the Yarrowia cells grow and can produce the desired biomass and/or compound of interest.

It is favourable if a process variable of the fermentation process, for which during the course of the fermentation process measured values can be determined approximately in real time, is selected as the process variable to be measured. For this reason, the variance between the process model and the course of the actual fermentation process can be determined approximately in real time, particularly if the process model is calculated in parallel with the ongoing actual fermentation process. A direct comparison between the measured value of the selected process variable and the estimated value calculated or predicted by the process model can thus be performed for the process variable. A near-instant intervention is thus also enabled if there is an error in the fermentation process.

Expediently the repetitions of steps (vii) to (ix) of the present process are performed, i.e., comparison between measured value and estimated value of the selected process variable. Here, comparisons of the variance with the predetermined threshold value and modification of the model variable of the process model when the threshold value is exceeded, are performed with the respective modified model variable at short intervals in time such as every 10 seconds. The process model can thus be rapidly adjusted, e.g., with the presence of biological variability or variability in the process control (e.g., differences in the raw materials, or fluctuations in the composition of the substrate) so that a correspondingly valid prediction of the fermentation process can be performed for the further course of the process. In case of errors in the bioprocess, a near-instant response can occur. A near-instant intervention allows damage to the bioprocess and/or to the equipment, for instance, to be avoided or prevented.

In one alternative embodiment of the invention, what is known as the respiratory quotient, also abbreviated to RQ, may be selected as a process variable that is measured during the course of the fermentation process. The respiratory quotient is a process variable in fermentation processes, which represents an indicator of the processes within a bioactive cell. The respiratory quotient describes a ratio of the CO₂ produced at a given time to the O₂ consumed at the same time. During the course of a fermentation process, the respiratory quotient can be measured very easily in real time, e.g., using what is known as off-gas analysis.

As an alternative or additionally, a concentration of the biomass and/or a concentration of the substrate can be selected as process variables, which are measured during the course of the fermentation process. The determination of the current measured values of the concentration of the biomass during the course of the fermentation process occurs, for instance, based on the electrical properties of the biomass. Current measured values for the concentration of the substrate (e.g., sugar etc.) can likewise be determined in the course of the fermentation process, such as based on spectroscopic properties using spectroscopy, in particular using reflection spectroscopy.

Furthermore, it is advantageous if what is known as a deterministic process model is employed to estimate the process variables of the fermentation process. With a deterministic approach for an illustration of a fermentation process as a model, the knowledge of the respective process-specific, biochemical processes inside and outside of the biomass or cells employed is converted into mathematical equations during the course of the fermentation process.

In a further preferred embodiment, the steps (vi) to (ix) are carried out by a computer implemented method, more preferably a computer implemented method using algorithms. The aid from a computer and algorithms is beneficial in that variations in process variables can be easily compared with data from historical fermentation processes, and that these historical data can be used for a correct interpretation of the variation in the process variable.

### EXAMPLES

### Materials and Methods

### Yeast Strain

Yarrowia lipolytica (wild type, W29, CLIB 89, CBS 7504, ATCC®20460) was maintained by shake-flask cultivation in YPD medium (10 g/L Bacto yeast extract, 20 g/L Bacto peptone, 20 g/L glucose). Culture aliquots were collected in stationary phase and stored at −80° C. in 2 mL sterile vials with 30% (v/v) glycerol. Stock cultures described above were used to inoculate precultures for all experiments.

### Culture Dry Weight Determination

Biomass dry weight concentrations were determined by filtering 10 ml of diluted (1:1) culture samples using preweighed nitrocellulose filters (PALL Corporation Supor®, 0.45 μm 47 mm PES). After filters were washed with 20-30 ml demineralized water, dried in a microwave oven at 320 W during 20 minutes and weighed. The increase in weight was measured. Duplicated measurements did not vary more than 3.5% throughout the cultivation (Postma et al., 1989).

### Gas Analysis

Bioreactor exhaust gas was cooled (2° C.) in a condenser and dried (Perma pure gas dryer), prior to analysis of oxygen and carbon dioxide concentrations using Servomex gas analysis equipment. Off-gas concentrations of CO₂ and O₂ were measured using an NGA 2000 analyzer

### Substrate and Metabolite Analysis

Extracellular concentrations of residual glucose and formic acid present in culture supernatants were analyzed via high performance liquid chromatography (HPLC). Samples were measured via HPLC analysis on an Agilent 1260 HPLC, equipped with a Bio-Rad HPX 87H column. Detection was performed by means of an Agilent refractive index detector and an Agilent 1260 VWD detector.

### Example 1. Formic Acid and Glucose Co-Feeding in Aerobic Chemostat Cultures Medium Preparation

Synthetic medium with vitamins (SM) was prepared as described previously (Verduyn et al., 1992). Briefly, SM included: 5 g/L (NH₄)₂SO₄, 3 g/L K₂HPO₄, 0.5 g/L Mg₂SO₄·(7 H₂O), 1 ml/L trace metals solution, 1 ml/L vitamins solution, containing 5 g/L of glucose as carbon source. For the continuous cultivations featuring fixed ratios of formic acid and glucose, relevant amounts of concentrated formic acid (99% w/w) were aseptically added to the autoclaved medium.

Shake flask cultivations were performed with Yarrowia lipolytica W29 in 500 mL flasks using 100 mL of synthetic medium as described above, in orbital shakers at 30° C. and 200 rpm.

### Formic Acid and Glucose Co-Feeding in Aerobic Chemostat Cultures

Aerobic, glucose-limited chemostat cultivations were performed in 2L laboratory bioreactors (Applikon Biotechnology, Delft, The Netherlands) thermostatically set at 30° C. The working volume was kept at 1 L using a peristaltic pump connected to a level sensor. The pH was kept at 5.0 via automatic addition of KOH 2M. The bioreactors were stirred at 800 rpm and sparged with air at a flow rate of 0.5 L/min. The dissolved oxygen concentration was monitored via an oxygen electrode, remaining above 30% throughout the cultivation. After the batch phase, the dilution rate (that in steady state equals the specific growth rate) was set at 0.1 h⁻¹. The relevant amount of formic acid was aseptically added to a medium vessel including all other components of the medium, and then fed into the bioreactors with a flow rate set at 100 mL/h. The concentrated formic acid stocks were prepared aseptically using concentrated formic acid (99% w/w, Merck) and autoclaved demineralized water.

A steady state was defined as the situation in which at least five volume changes had passed since the last change in dilution rate and in which the biomass concentration, as well as the CO₂ concentration in the exhaust gas remained constant (<3% variation) for at least two volume changes.

Table 1 shows the concentrations of formic acid and glucose fed to the different chemostat cultivations that were performed.

TABLE 1
Concentrations and ratio of formic acid and glucose in chemostat
experiments, which were determined by HPLC analysis after
sampling from the medium reservoir connected to the bioreactor.

| | Ingoing medium composition | | |
|---|---|---|---|
| | Formic acid (F) | Glucose (G) | Ratio F:G |
| No. | mM | mM | mol/mol |
| 1 | 0.0 | 29.2 | 0 |
| 2 | 0.0 | 28.0 | 0 |
| 3 | 0.2 | 28.2 | 0 |
| 4 | 82.6 | 28.1 | 2.9 |
| 5 | 136.0 | 28.1 | 4.8 |
| 6 | 140.6 | 28.2 | 5.0 |
| 7 | 142.2 | 28.6 | 5.0 |
| 8 | 188.2 | 27.2 | 6.9 |
| 9 | 217.2 | 28.0 | 7.8 |
| 10 | 225.9 | 27.8 | 8.1 |
| 11 | 226.3 | 27.7 | 8.2 |
| 12 | 256.9 | 27.9 | 9.2 |
| 13 | 257.4 | 27.7 | 9.3 |
| 14 | 323.3 | 28.0 | 11.5 |
| 15 | 322.4 | 27.9 | 11.6 |

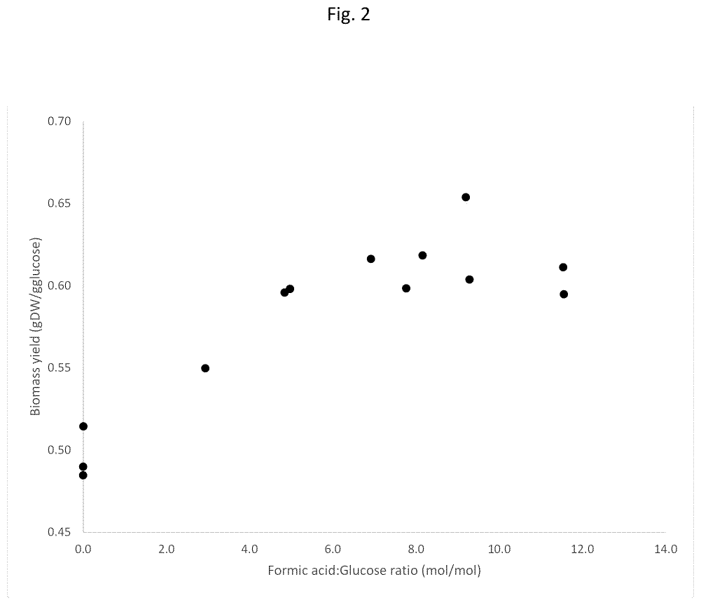

Once the cultures reached steady state, samples were taken and the biomass yield on glucose was calculated. The results in FIG. 2 show that adding formic acid to the medium increased the biomass yield, and at a molar ratio of formic acid to glucose from 5 to 12 the biomass yield was between 0.6 to 0.67 gDW/gGlucose. The fact that the biomass yield does not substantially increase when the formic acid to glucose ratio is increased from 5 to 12 indicates that the benefit of adding more formic acid decreases from a formic acid to glucose ratio of 5 onwards, and that increasing the ratio beyond 12 leads to a waste of formic acid. Also, a ratio of formic acid to glucose beyond 12 leads to formic acid accumulation in the broth, which becomes toxic to the cells.

The residual concentrations of formic acid in the continuous culture remained below 6 mM (data not shown), which indicated that the majority of the added formic acid was consumed by the Yarrowia cells, resulting in a linear increase of the biomass specific uptake rate of formic acid.

### Example 2. Formic Acid Glucose Co-Feeding in Aerobic Fed Batch Cultures Medium Preparation

Tables 2A and 2B show the compositions of the media for the preculture and the 10 L fed batch fermentations.

The vitamin stock solution contained (g/kg): Biotin, 0.050; CaD(+) panthotenate, 1.0; nicotinic acid, 1.0; myo-inositol, 25.0; thiamine hydrochloride, 1.0; Pyridoxine hydrochloride, 1.0; p-aminobenzoic acid, 0.20.

The trace elements stock solution contained H₂SO₄ (98%) (5 ml/kg) and further in (g/kg): zinc sulfate.7H₂O, 4.5; manganese (II) chloride.2H₂O, 0.84; cobalt (II) chloride.6H₂O, 0.30; copper (II) sulfate.5H₂O, 0.30; di-sodium molybdate.2H₂O, 0.40; iron sulfate.7H₂O, 3.0; boric acid, 1.0; potassium iodide, 0.10.

TABLE 2A
Preculture medium

| | Final |
|---|---|
| | concentration |
| Compound | (g/kg) |
| K₂SO₄ | 6.6 |
| KH₂PO₄ | 3.0 |
| MgSO₄•7H₂O | 0.5 |
| Urea * | 2.3 |
| Glucose * | 50 |
| CaCl₂•2H₂O * | 0.0045 |
| Trace Elements stock sol. | 1 |
| Vitamin stock sol. | 1 |

* Urea, calcium chloride and glucose were added as a stock solution to achieve the final concentration as described in the table.

TABLE 2B
Batch medium 10 L bioreactor

| Compound | Concentration | Unit |
|---|---|---|
| Glucose * | 5 | g/kg based on fermenter |
| | | start mass |
| (NH₄)₂SO₄ | 1.83 | g/kg based on fermenter |
| | | start mass |
| Basildon | 0.5 | g/kg based on fermenter |
| | | start mass |
| KH₂PO₄ | 16.0 | g/kg based on fermenter |
| | | end mass |
| MgSO₄•7H₂O | 8.0 | g/kg based on fermenter |
| | | end mass |
| CaCl₂•2H₂O * | 0.0945 | g/kg based on fermenter |
| | | end mass |
| Trace Elements | 21 | g/kg based on fermenter |
| stock sol. | | end mass |
| Vitamin stock sol. | 11 | g/kg based on fermenter |
| | | end mass |

* Calcium chloride and glucose were added as a stock solution to achieve the final concentration as described in the table.

The solutions for pH correction and foam remediation were NH₃ (25 w/w %), 2 M H₂SO₄ and Basildon 86-013.

### Preculture

Precultures were incubated in flat bottom flaks with baffles at 150 rpm, at 30° C.; 0.4 mL of Yarrowia cell stock was added to 400 mL of preculture medium as shown in Table 2A. After 26 h of incubation the contents of the flasks were transferred to 10L fermenters for the fed batch fermentation. The OD600 at the end of the preculture incubations in flask 1, 2 and 3 was 3.8, 3.9 and 4.8, respectively

### Fed Batch Fermentation Process

Each vessel contained 3.6 kg of batch medium as disclosed in Table 2B. 400 g of preculture of flask 1, 2 and 3 as described above was used to inoculate a fed batch with a formic acid to glucose ratio of 0:1, 3:1 and 5:1 respectively, as described below. The process started with a batch phase until carbon depletion. The oxygen uptake rate (OUR) and carbon dioxide production rate (CPR) were monitored. When the OUR showed a sharp drop, the batch phase was finished and the carbon feed of formic acid and glucose was started. The feed of formic acid and glucose having a composition of formic acid: glucose ratio of 0:1, 3:1 or 5:1 mol formic acid/mol glucose as shown in Table 3 was sterilized at 121° C. for 20 minutes. The formic acid was added to the solution in an aseptic way.

TABLE 3
Composition of the feed with a formic acid to glucose ratio
(F:G) of 0:1, 3:1 and 5:1 mol formic acid/mol glucose

| | Ratio F:G (mol/mol) | | |
|---|---|---|---|
| | 0:1 | 3:1 | 5:1 |
| Compound | Concentration (g/kg) | | |
| Glucose monohydrate | 275 | 275 | 275 |
| Water | 725 | 533 | 405 |
| Formic acid | 0 | 192 | 320 |

The feed profile consisted of an exponential phase starting at 8 g/h feed solution that increased exponentially with an exponent of 0.2 h⁻¹, followed by a constant phase that started when 100 g of pure glucose had been dosed (including the glucose in the batch medium). The constant feed phase commenced 10 hours after feed start. At the end of the fermentations, about 3.3 kg of feed had been added to the three fed batch fermentations.

During the fermentations the temperature was controlled at 30° C. and pH 4.9. The dissolved oxygen concentration was controlled at >20% of saturation at atmospheric conditions by the agitation speed, and when the maximum agitation speed was achieved by the airflow. The fermentations were stopped after 75h of incubation

Tables 4 to 6 show that biomass cell dry weight (CDW) increased steadily in the three fermentations with formic acid: glucose ratio of 0:1, 3:1 and 5:1 mol/mol, respectively. The glucose levels started around 10 g/l and decreased rapidly and the formic acid concentrations in Tables 5 and 6 remained <<1 g/l for all cultures, which confirmed that the culture was growing carbon-limited. The biomass yield on glucose was determined as the cumulative amount of biomass formed, divided by the cumulative amount of glucose consumed in the fermentation process.

TABLE 4
Biomass cell dry weight of Yarrowia lipolytica in a
fed batch fermentation with a feed of formic acid and
glucose at a formic acid:glucose ratio of 0:1 mol/mol

| | Biomass, | Glucose, | Formic acid, |
|---|---|---|---|
| Age, h | g/kg | g/l | g/l |
| 0.0 | n.d. * | 10 | n.a. |
| 3.6 | n.d. | 10 | n.a. |
| 8.7 | n.d. | n.a. ** | n.a. |
| 11.1 | 10.5 | n.a. | n.a. |
| 21.2 | n.d. | n.a. | n.a. |
| 26.4 | 21.9 | n.a. | n.a. |
| 44.7 | 46.0 | n.a. | n.a. |
| 49.6 | n.d. | n.a. | n.a. |
| 68.2 | 61.1 | n.a. | n.a. |
| 74.2 | 65.6 | n.a. | n.a. |

* n.d.: not determined;
** n.a.: under the detection limit

TABLE 5
Biomass cell dry weight of Yarrowia lipolytica in a fed
batch fermentation with a feed of formic acid and glucose
at a ratio of formic acid:glucose ratio of 3:1 mol/mol

| | Biomass, | Glucose, | Formic acid, |
|---|---|---|---|
| Age, h | g/kg | g/l | g/l |
| 0.0 | n.d. | 10.2 | n.d. |
| 3.6 | n.d. | 9.98 | n.d. |
| 11.4 | 10 | n.a. * | 0.12 |
| 21.2 | n.d. | 0.03 | n.d. |
| 26.4 | 23 | 0.05 | 0.11 |
| 44.7 | 51 | 0.06 | n.d. |
| 49.6 | n.d. | 0.05 | 0.07 |
| 68.2 | 69.2 | 0.07 | n.d. |
| 74.2 | 73.4 | 0.08 | 0.10 |

* n.d.: not determined;
** n.a. under the detection limit

TABLE 6
Biomass cell dry weight of Yarrowia lipolytica in a fed
batch fermentation with a feed of formic acid and glucose
at a ratio of formic acid:glucose ratio of 5:1 mol/mol

| | Biomass, | Glucose, | Formic acid, |
|---|---|---|---|
| Age, h | g/kg | g/l | g/l |
| 0.0 | n.d. | 10 | n.d. |
| 3.6 | n.d. | 10.1 | n.d. |
| 9.7 | 10 | n.a. * | 0.10 |
| 19.9 | n.d. | 0.22 | n.d. |
| 26.4 | 28 | 0.26 | 0.22 |
| 44.7 | 57 | 0.35 | 0.18 |
| 68.2 | 79.3 | 0.38 | n.d. |
| 74.2 | 78.8 | 0.40 | 0.23 |

* n.d.: not determined;
** n.a.: under detection limit

The results in Table 7 show that the yield of biomass on consumed glucose was highest at a formic acid: glucose ratio of 5:1 mol/mol.

TABLE 7
Yield of biomass formed on glucose consumed of
Yarrowia lipolytica on consumed glucose when
fed formic acid and glucose at different ratio's

| | Yield of |
|---|---|
| | produced |
| | biomass on |
| | consumed |
| Formic acid:glucose ratio | glucose, |
| mol/mol | g/g |
| 0:1 | 0.44 |
| 3:1 | 0.47 |
| 5:1 | 0.50 |

1. A process for fermenting Yarrowia in a bioreactor, comprising
cultivating Yarrowia in a medium, and
adding a feed comprising formic acid and a sugar to the medium in a molar ratio of formic acid to sugar of 1 to 13 mol formic acid/mol monosaccharide.

2. The process according to claim 1, wherein the sugar comprises glucose, fructose, sucrose, maltose, maltotriose and/or another oligosaccharide.

3. The process according to claim 1, wherein the feed has a pH of 1 to 6.

4. The process according to claim 1, wherein the process for fermenting Yarrowia is performed in a continuous culture or a fed batch culture.

5. The process according to claim 4, wherein the continuous culture has a dilution rate of 0.03 h⁻¹ to 0.3 h⁻¹ during steady state.

6. The process according to claim 4, wherein the fed batch culture has a feed rate profile comprising an exponentially increasing feed phase and a constant feed phase.

7. The process according to claim 6, wherein during the exponentially increasing feed phase Yarrowia has a biomass specific growth rate ranging from 0.03 h 1 to 0.3 h⁻¹.

8. The process according to claim 1, wherein the process for fermenting Yarrowia is performed under carbon limited conditions.

9. The process according to claim 1, wherein the process for fermenting Yarrowia comprises producing Yarrowia and/or a polypeptide and/or a compound of interest.

10. The process according to claim 1, wherein the Yarrowia comprises at least one polynucleotide coding for a polypeptide or at least one polynucleotide coding for a polypeptide involved in production of a compound of interest by the cell.

11. The process according to claim 1, wherein Yarrowia is Yarrowia lypolytica.

12. The process according to claim 1, further comprising
i) capturing CO₂ from the bioreactor;
ii) reducing the CO₂ to formic acid in a reduction unit; and
iii) feeding at least a part of the formic acid from the reduction unit into the bioreactor.

13. The process according to claim 12, further comprising
iv) electrolyzing water into H₂ and O₂ in an electrolysis unit, and feeding at least a part of the H₂ into the reduction unit for reducing the CO₂ to the organic feedstock, and optionally comprising
v) feeding at least a part of the O₂ into the bioreactor.

14. The process according to claim 1, comprising controlling the process comprising:
(vi) selecting at least one process variable of the process, for which current measured values are determined during a course of the process;
(vii) comparing a respective current measured value of the at least one selected process variable with a corresponding estimated value of a model variable estimated by a process model for said at least one process variable;
(viii) comparing a variance between the respective current measured value and the corresponding estimated value for the at least one selected process variable with a predetermined threshold value; and
(ix) changing at least one defined model variable employed in the process model when the predetermined threshold value is exceeded by the variance:
wherein (vi) to (ix) with a respective changed model variable are executed until the variance is placed below a predetermined threshold value; and optionally
wherein in a case that, after a predetermined number of repetitions of (i) to (iv), the threshold value is met by the variance, the process is discontinued and a warning is generated as output.

15. A computer implemented method for controlling the process as defined in claim 14.
